(19) United States
(12) Patent Application Publication
(10) Pub. No.: US 2011/0011397 A1
(43) Pub. Date: Jan. 20, 2011

(54) MULTIPLE CHAMBERS MASK

(75) Inventors: Hedi Ziv, Burwood (AU); Rikus Slabbert, Victoria (AU); Conor McCooey, Brighton (AU)

Correspondence Address: SUGHRUE MION, PLLC, 2100 Pennsylvania Avenue, N.W., Washington, DC, 20037, US

(73) Assignee: COMPUMEDICS MEDICAL INNOVATION PTY LTD., Abbotsford (AU)

(21) Appl. No.: 12/831,175

(22) Filed: Jul. 6, 2010

(30) Foreign Application Priority Data
Jul. 6, 2009 (AU) AU2009903135

(51) Int. Cl. A61M 16/00; A61M 16/01

(52) U.S. Cl. 128/203.12; 128/207.18

(57) ABSTRACT

The present invention provides methods and apparatuses for gas delivery, in particular, a mask including at least one porous barrier defining at least two chambers in gaseous communication. The porous barrier may include a plurality of pores, which define a tapered shape. The mask may include a pliable nasal cushion. The invention provides a method for providing pressurised gas to a subject by supplying gas from a pressurised source to a mask, transmitting the gas into a first chamber at a first pressure, transmitting the gas into a second chamber at a second pressure through a plurality of pores, and transmitting the gas from the second chamber through a mask exit.

### CROSS REFERENCE TO RELATED APPLICATION

This application claims priority from Australian Provisional Patent Application No. 2009903135, filed Jul. 6, 2009 in the Australian Patent Office, the entire disclosure of which is incorporated herein by reference.

### FIELD OF THE INVENTION

The present invention relates to masks for the provision of gases to subjects, in particular, masks for delivery of pressurised gas to the airways of a subject.

### BACKGROUND

There are a number of treatments that require the use of a mask for the delivery of gas or air to a subject. For example, in the treatment of sleep apnoea, gas is often delivered at continuous positive airway pressure (CPAP) wherein gas is supplied continuously at a pressure greater than ambient, or variable positive airway pressure (VPAP) wherein gas is supplied at varying pressures, or other such methods such as BiPAP wherein gas is supplied at two pressures, or APAP wherein gas is supplied at pressures determined automatically by the delivery system to a sleeping subject through a mask to keep the patient's airways open for effective respiration. Often gas must be delivered through a mask for sustained time periods, for example, through a whole overnight period of sleep.

It is important for continuous therapeutic benefit from pressurised gas or air that a mask assembly be comfortable and relatively leak-proof when positioned for gas delivery. The mask must be comfortable so that a subject achieves the therapeutic benefit of relatively unbroken sleep periods. Masks known in the art often incorporate an interface such as a cushion or pillow, made of soft material to be most comfortable for a subject during long periods of use, but are capable of sealing adequately to minimise leaks. A nasal cushion, for example, is sealingly engaged with the bridge and sides of the nose, and the upper lip to provide a leak-proof conduit for pressurised gas delivery.

Prior art mask assemblies have been designed to allow some gas leakage from the mask assembly during gas delivery. An unfortunate consequence is the acoustic noise attributable to these leaks may cause a subject to have difficulty falling asleep and then being aroused from sleep during positive airway pressure treatment Arousals, in turn, may limit the efficacy of the treatment. Further, the acoustic noise also may affect the sleeping of nearby persons. What is needed is an interface for pressurised gas delivery in any of CPAP/VPAP/BiPAP/APAP system wherein the interface delivers gas to the airways of a subject with reduced acoustic noise.

### SUMMARY OF THE INVENTION

It is an object of the invention to provide an interface for a gas-delivery mask that generates reduced acoustic noise during gas delivery. The present invention most advantageously provides solutions to the problem of noisy masks when pressurised air or gases is provided to a subject. The mask is both comfortable and less noisy than masks currently found in the art. The invention provides an interface for a mask for delivery of pressurised gas to a subject incorporating means to reduce the acoustic noise generated from the pressurised gas. The interface may be a nasal cushion or other means, such as a face cushion. The noise reduction means comprises of a baffling means. The baffling means may comprise a porous barrier defining volumes in gaseous communication within the nasal interface. In one embodiment, there are two adjacent volumes defined by a porous barrier.

Surprisingly, the acoustic noise of multiple adjacent volumes is reduced compared with a single volume. The invention has many possibilities for uses in different applications whenever pressurised gas is delivered to a subject. Some examples are for delivering oxygen in aircraft, or for delivering gaseous anaesthetics or therapeutic substances suitable for gas delivery. In particular, the invention will be most advantageously used for gas delivery for application of CPAP therapy. The reduced acoustic noise generated by a gas-delivery mask according to the invention enables a more efficient sleep with fewer arousals.

In one aspect, the invention provides a mask comprising of a gas supply, a gas output, and at least one porous barrier defining at least two chambers in gaseous communication. In one embodiment, the mask incorporates a first chamber in gaseous communication with the gas supply and the second chamber, the communication being through apertures for gaseous flow into the ambient air. In another aspect, the invention provides a mask for delivering pressurised gas to a subject, the mask comprising a first aperture in gaseous communication with a pressurised gas supply, a second aperture for efflux of pressurised gas, and at least one porous barrier defining at least two chambers, wherein the chambers are in gaseous communication. The mask may include a conduit in communication with the gas supply and the mask. The porous barrier may includes a plurality of apertures in communication with the chambers. The apertures may define a tapered shape. In one embodiment, the mask includes a nasal cushion. In another embodiment, the nasal cushion may comprise a pliable material.

In another aspect, the invention provides a method of providing pressurised gas to a subject, comprising the steps of supplying gas from a pressurised source to a mask, transmitting the gas into a first chamber at a first pressure, transmitting the gas into a second chamber at a second pressure, and transmitting the gas from the second chamber through an exit. In one aspect, the step of transmitting the gas to second chamber or through the exit occurs through a plurality of apertures. In another aspect, the invention provides a mask comprising of a first chamber for engagement with the face of a subject, a second chamber in gaseous communication with the first chamber, and a porous baffle defining the boundary between the chambers.

### BRIEF DESCRIPTION OF THE FIGURES

FIG. 1 shows a longitudinal transverse view through a mask assembly having a nasal cushion interface according to the invention.

FIG. 2 shows a transverse view of a few slot leak aperture design of a mask assembly having a nasal cushion according to the invention.

FIG. 3 shows a transverse view of multiple leak apertures design of a mask assembly having a nasal cushion according to the invention.

FIGS. 4a and 4b show a comparison between a parallel wall pore (a) and a tapered pore (b).

### DETAILED DESCRIPTION OF THE INVENTION

The invention advantageously exploits the acoustic muffling effect of adjacent chambers in gaseous communication, the chambers defined by a baffling means. The baffle means comprises of a barrier incorporating pores of defined cross-sectional areas. The baffle may incorporate a plurality of small pores which cooperate to decrease the acoustic noise attributable to the gas flow. The pores may also be tapered. The pores may be frusto-conical in shape. The baffling means may comprise of multiple small tapered pores. Surprisingly, the acoustic noise of the multiple small tapered pores is reduced compared with fewer, larger pores creating the same total cross-sectional area. The acoustic noise of the tapered pores or frusto-conical pores may be reduced compared with cylindrical pores. The reduced acoustic noise enables a more efficient sleep with fewer arousals.

It is known in the art that for two pores in series design, compared to a single pore design, having the same overall pressure drop at the same flow rate, the two pores should have a diameter of about 2^{1/4} d=1.19 d, where d is the diameter of a single pore. The velocity of gas movement through the pores reduces to 8\sqrt{2} V where V is the original mean velocity. Given that the sound power is proportional to the product of pore area and velocity to the 8^{th} power, the acoustic power emission per pore should be reduced by a factor of 8\sqrt{2}=11.3 or about 10 dB. In practice, the internal pore noise is not fully absorbed. A further advantage is that the reduction in velocity attributable to chambers in gaseous communication would be expected to reduce the external draught effect at the leak site.

The pressure difference of gas in volumes in gaseous communication is proportional to the cross-sectional area defined by the apertures of the pores. Optimal noise suppression may be effected by reducing the magnitude of pressure drop, by introducing intermediate pressure drops in multiple intermediate chambers, and implementing multiple small tapered pores, although either feature may be incorporated in an embodiment to achieve some noise suppression.

The figures illustrate embodiments of the invention. It will be understood that there are many other possible embodiments of the invention and that the invention is limited only by the scope of the claims appended hereto.

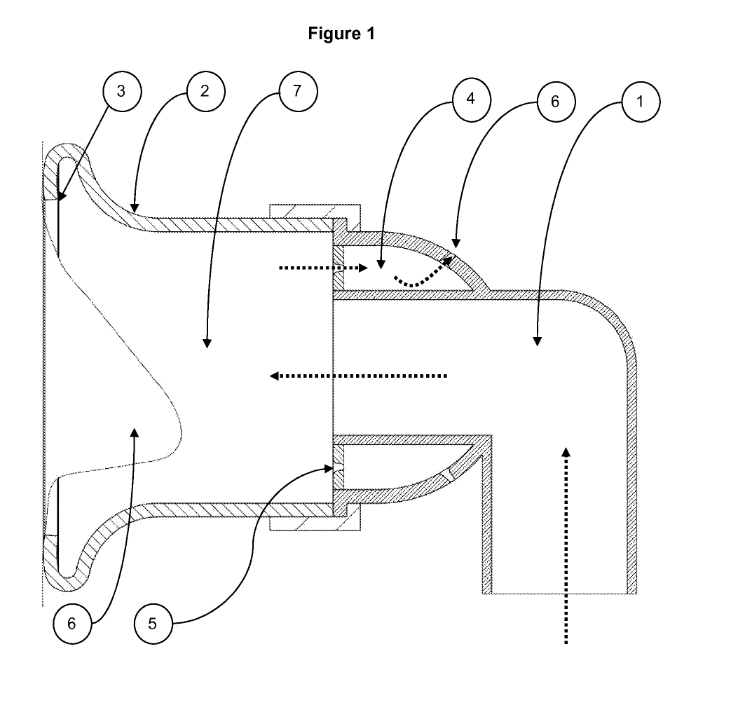

FIG. 1 shows an embodiment of the invention as a dual chamber gas-delivery mask. The left side of the drawing shows a nasal mask in operative position on the face of a subject, with the subject's nose (6) extending into a first chamber (7) defined by the nasal interface. In this embodiment, the interface is a nasal cushion (2). Alternative embodiments such as facial cushions or pillows may also be used. The PAP (positive airway gas pressure) is supplied via a hose (1) into the nasal cushion defining a first chamber (7), the nasal cushion engaging the subject's face with a flexible sealing membrane (3). The pressure differential between the above-ambient pressure gas from the hose (1) flowing into the nasal chamber (7) and a second chamber (4) in gaseous communication with the first chamber (7) results in gas flux between the chambers.

A further gas pressure differential between the second chamber (4) and the ambient air results in gas flux into the air. The pressurised gas from the second chamber (4) flows to the ambient air via pores (6) which may be tapered or frusto-conical in shape, but may also take other shapes as necessary, such as being cylindrical. The gas may flow through pores (5) in a porous baffle defining the boundary between the chambers (7),(4). FIG. 1 shows two chambers in gaseous communication but further chambers may be incorporated into the mask. The invention includes more than two chambers, if necessary, according to the application. The pathway of the PAP through the supply hose and interface is shown by the dotted lines in FIG. 1. The total area of pores (5) between the main chamber (7) and the secondary chamber (4); and the total area of pores (6) between the secondary chamber (4) and the ambient air determines the pressure difference between the main chamber (7) and the secondary chamber (4), as well as the pressure difference between the secondary chamber (4) and the ambient air. The reduction of pressure difference between the two chambers or the secondary chamber and the ambient air, generates a lower flow through the pores and thus creates less turbulence, resulting in a reduced acoustic noise level.

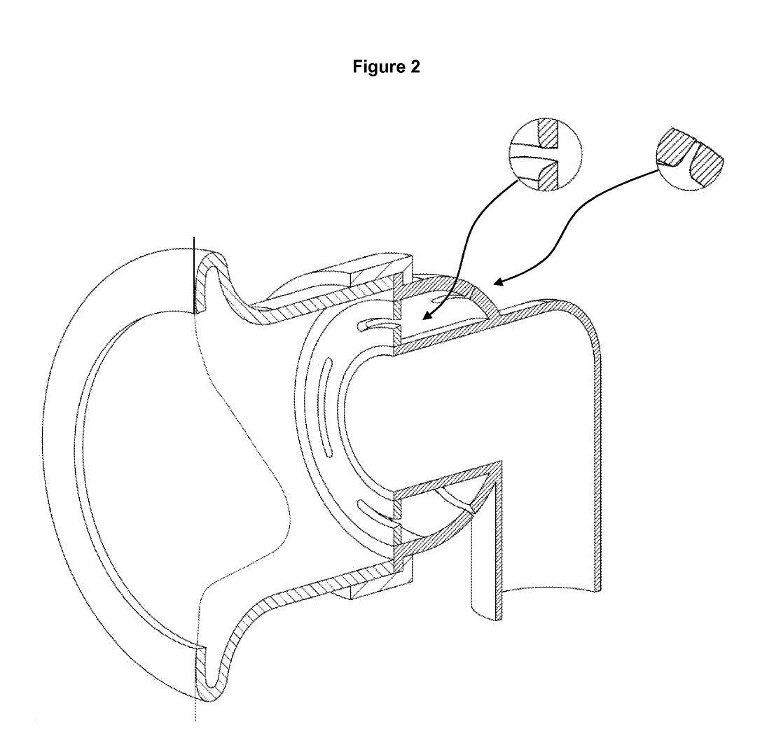

FIG. 2 shows an embodiment of the invention having multiple large pores for the gas leak.

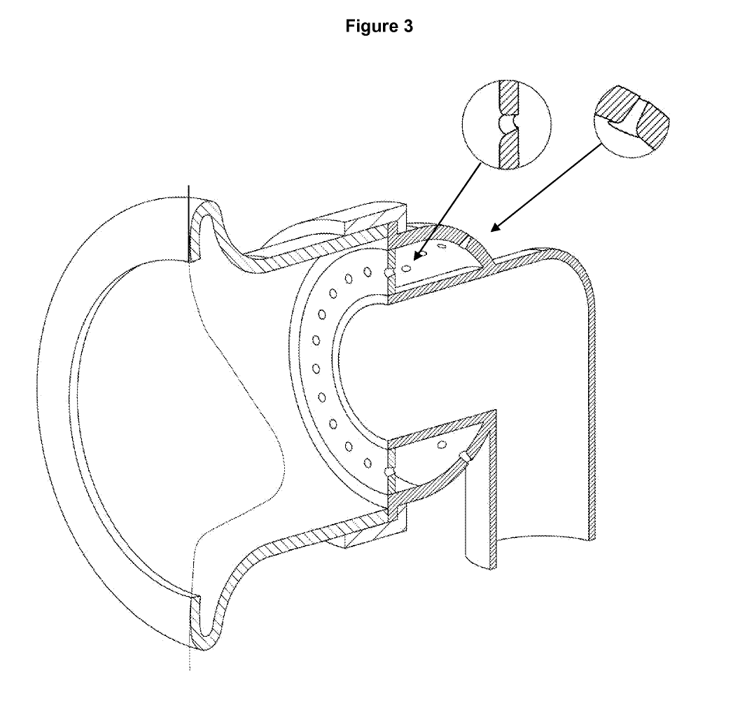

FIG. 3 displays small multiple tapered pores for the gas leak.

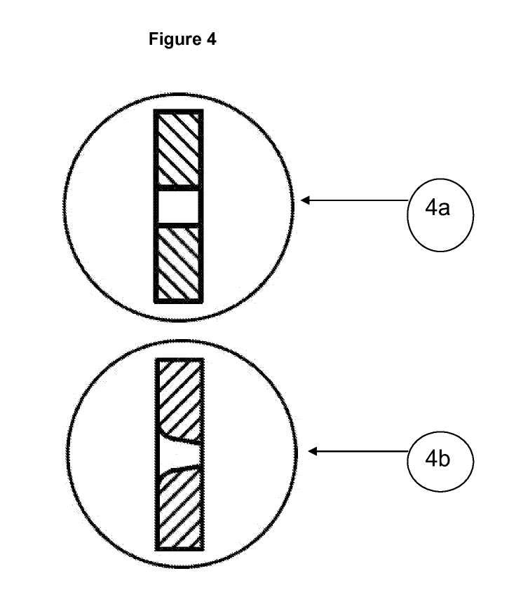

FIG. 4a shows an embodiment of the invention having a cylindrical pore. FIG. 4b a further embodiment having a tapered pore. The smooth entrance to the aperture reduces turbulence of the gas being released; the tapered shape decelerates the gas flow, resulting in a reduced acoustic noise level.

We claim:

1. A mask comprising:
a gas supply;
a gas output; and
at least one porous barrier between at least two chambers in gaseous communication.

2. The mask of claim 1, further comprising a conduit in communication with the gas supply and the mask.

3. The mask of claim 1, wherein the porous barrier includes a plurality of pores in communication with said chambers.

4. The mask of claim 3, wherein the pores define a tapered or frusto-conical shape.

5. The mask of claim 1, further comprising a nasal cushion.

6. The mask of claim 1, wherein the at least two chambers further comprise a first chamber is in gaseous communication with the gas supply and a second chamber including pores in gaseous communication with ambient air.

7. A mask delivering pressurised gas to a subject, the mask comprising:
a first aperture in gaseous communication with a pressurised gas supply;
a second aperture for efflux of pressurised gas; and
at least one porous barrier defining at least two chambers, wherein the chambers are in gaseous communication.

8. The mask of claim 7, further comprising a conduit in communication with the gas supply and the mask.

9. The mask of claim 7, wherein the porous barrier includes a plurality of pores in communication with said chambers.

10. The mask of claim 9, wherein the pores define a tapered or frusto-conical shape.

11. The mask of claim 7, further comprising a nasal cushion.

12. The mask of claim 7, wherein the at least two chambers further comprise a first chamber is in gaseous communication with the gas supply and a second chamber including pores in gaseous communication with ambient air.

13. A mask comprising:
a first chamber engaging with at least a portion of a face of a subject;
a second chamber in gaseous communication with said first chamber; and
a porous baffle between the first chamber and second chamber.

14. The mask according to claim 13, wherein the porous baffle comprises tapered or frusto-conical pores.

15. The mask according to claim 13, wherein the first chamber comprises a pliable material.

16. The mask according to claim 13, operable in gaseous delivery of anaesthetics.

17. A method of providing pressurised gas to a subject, the method comprising the steps of:
supplying gas from a pressurised source to a mask;
transmitting the gas into a first chamber at a first pressure;
transmitting the gas into a second chamber at a second pressure; and
transmitting the gas from the second chamber through an exit.

18. The method of claim 17, wherein transmitting the gas to the second chamber or through the exit occurs through a plurality of pores.

19. The method of claim 17, wherein the gas incorporates an anaesthetic substance.

20. The method of claim 17, wherein the gas incorporates a therapeutic substance.

21. The method of claim 17, wherein the gas includes oxygen at a concentration greater than ambient.
